(19) United States
(12) Patent Application Publication
(10) Pub. No.: US 2024/0148287 A1
(43) Pub. Date: May 9, 2024

(54) Biological Information Measurement Device And Biological Information Measurement Method

(71) Applicant: SEIKO EPSON CORPORATION, Tokyo (JP)

(72) Inventors: Akira IKEDA, Chino (JP); Atsushi MATSUO, Azumino (JP)

(21) Appl. No.: 18/504,361

(22) Filed: Nov. 8, 2023

(30) Foreign Application Priority Data
Nov. 9, 2022 (JP) 2022-179372

(51) Int. Cl. A61B 5/1455; A61B 5/00

(52) U.S. Cl. CPC A61B 5/14551; A61B 5/7221; A61B 5/7225; A61B 5/742

(57) ABSTRACT

A biological information measurement device includes: a light-emitting unit including a first light-emitting element that emits red light and a second light-emitting element that emits infrared light; a light-receiving unit configured to receive the red light emitted from the first light-emitting element and the infrared light emitted from the second light-emitting element and generate a first light-receiving signal based on the red light and a second light-receiving signal based on the infrared light; and a controller configured to calculate an oxygen saturation concentration. The controller is configured to calculate the oxygen saturation concentration using the first light-receiving signal and the second light-receiving signal, calculate correlation data using the first light-receiving signal and the second light-receiving signal, and determine the oxygen saturation concentration based on the correlation data.

The present application is based on, and claims priority from JP Application Serial Number 2022-179372, filed Nov. 9, 2022, the disclosure of which is hereby incorporated by reference herein in its entirety.

### BACKGROUND

### 1. Technical Field

The present disclosure relates to a biological information measurement device and a biological information measurement method.

### 2. Related Art

A measurement device that non-invasively measures biological information of a subject is known. The measurement device described in JP-A-2022-86227 measures a pulse wave and an oxygen saturation concentration. The measurement device includes a first light emitter, a second light emitter, and a third light emitter. The first light emitter emits green light having a green wavelength band to a measurement site. The second light emitter emits red light having a red wavelength band to the measurement site. The third light emitter emits near infrared light having a near infrared wavelength band to the measurement site. The measurement device specifies the oxygen saturation concentration by analyzing a detection signal representing a light-receiving intensity of the red light and a detection signal representing a light-receiving intensity of the near infrared light. The measurement device specifies the oxygen saturation concentration using a pulsation component of an artery.

The detection signal representing the light-receiving intensity of the red light and the detection signal representing the light-receiving intensity of the infrared light fluctuate depending on body movement or the like of the subject. When the detection signal fluctuates, the measurement accuracy of the oxygen saturation concentration specified based on the detection signal fluctuates, but it is difficult to determine the measurement accuracy.

### SUMMARY

A biological information measurement device according to the present disclosure includes: a light-emitting unit including a first light-emitting element that emits red light and a second light-emitting element that emits infrared light; a light-receiving unit configured to receive the red light emitted from the first light-emitting element and the infrared light emitted from the second light-emitting element and generate a first light-receiving signal based on the red light and a second light-receiving signal based on the infrared light; and a controller configured to calculate an oxygen saturation concentration. The controller is configured to calculate the oxygen saturation concentration using the first light-receiving signal and the second light-receiving signal, calculate correlation data using the first light-receiving signal and the second light-receiving signal, and determine the oxygen saturation concentration based on the correlation data.

A biological information measurement method according to the present disclosure includes: emitting red light and infrared light to a subject; receiving the red light and the infrared light passing through the subject; generating a first light-receiving signal based on the received red light and a second light-receiving signal based on the received infrared light; calculating an oxygen saturation concentration using the first light-receiving signal and the second light-receiving signal; calculating correlation data based on the first light-receiving signal and the second light-receiving signal; and determining the oxygen saturation concentration based on the correlation data.

### BRIEF DESCRIPTION OF THE DRAWINGS

FIG. 1 is a diagram showing a schematic configuration of a measurement device.

FIG. 2 is a diagram showing a schematic configuration of a measurement surface.

FIG. 3 is a diagram showing a block configuration of the measurement device.

FIG. 4 is a diagram schematically showing a detection signal.

FIG. 5 is a diagram showing a relationship between a frequency and a signal intensity of each detection signal in a predetermined time.

FIG. 6 is a diagram showing red light detection signal data and infrared light detection signal data.

FIG. 7 is a diagram showing red light detection signal data and infrared light detection signal data.

FIG. 8 is a flowchart for determining an oxygen saturation concentration.

FIG. 9 is a graph showing a measurement result of the oxygen saturation concentration.

FIG. 10 is a graph showing a measurement result of the oxygen saturation concentration.

FIG. 11 is a diagram showing a block configuration of the measurement device.

FIG. 12 is a diagram showing a relationship between a frequency and a signal intensity of each detection signal in a predetermined time.

FIG. 13 is a flowchart for determining an oxygen saturation concentration.

### DESCRIPTION OF EMBODIMENTS

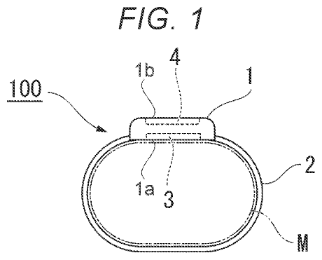

FIG. 1 shows a schematic configuration of a measurement device 100. FIG. 1 is a side view of the measurement device 100. The measurement device 100 non-invasively measures biological information of a user M such as a human. The measurement device 100 is a watch type portable device worn on a measurement site of the user M. The measurement device 100 shown in FIG. 1 is worn on, for example, the wrist of the user M.

The measurement device 100 measures the biological information of the user M. The measurement device 100 measures a pulse wave including a pulse interval or the like and an oxygen saturation concentration as the biological information. The pulse wave interval is represented as a post pacing interval (PPI). The oxygen saturation concentration is represented as SpO₂. The pulse wave indicates a change with time in a volume of a blood vessel in conjunction with the heart beat. The oxygen saturation concentration indicates a proportion of hemoglobin bound to oxygen to hemoglobin in arterial blood of the user M. The oxygen saturation concentration is an index for evaluating a breathing function of the user M. The measurement device 100 may measure the biological information other than the pulse and the oxygen saturation concentration. The measurement device 100 measures, for example, a glucose concentration in the arterial blood and an alcohol concentration in the arterial blood. The measurement device 100 corresponds to an example of a biological information measurement device. The user M corresponds to an example of a subject. The measurement device 100 includes a housing 1 and a belt 2. The housing 1 accommodates a detection unit 3 and a display panel 4.

The housing 1 is an exterior housing accommodating a unit or the like provided in the measurement device 100. The housing 1 has a measurement surface 1a and a display surface 1b. The measurement surface 1a is a surface facing the measurement site of the user M. The measurement surface 1a comes into contact with the measurement site of the user M. The display surface 1b is a surface visible to the user M. In addition to the detection unit 3 and the display panel 4, the housing 1 accommodates a control unit 30, a memory 40, or the like, which will be described later.

The belt 2 is a member that is used when the user M wears the housing 1 on the measurement site. The belt 2 is attached to, for example, a side surface of the housing 1. The belt 2 is wound around the measurement site, and thereby the housing 1 is worn on the measurement site of the user M.

The detection unit 3 is disposed on the measurement surface 1a of the housing 1. The detection unit 3 is disposed at a position facing the measurement site of the user M. The detection unit 3 acquires various types of information used when measuring the biological information.

The display panel 4 is disposed on the display surface 1b of the housing 1. The display panel 4 is visible to the user M. The display panel 4 displays various types of the measured biological information. The display panel 4 may display information such as a reliability index of the biological information and time other than the biological information. The display panel 4 corresponds to an example of a display unit.

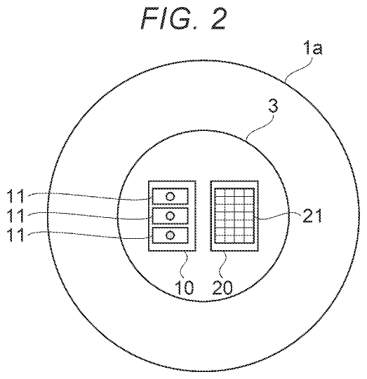

FIG. 2 shows a schematic configuration of the measurement surface 1a. FIG. 2 shows the schematic configuration of the measurement surface 1a when viewed from the outside. The measurement surface 1a shown in FIG. 2 is formed in a circular shape, but is not limited thereto. The measurement surface 1a may be formed in various shapes such as a square shape and an elliptical shape. The detection unit 3 is disposed on the measurement surface 1a. The detection unit 3 includes a light-emitting element unit 10 and a light-receiving element unit 20.

The light-emitting element unit 10 emits light toward the measurement site of the user M. The light-emitting element unit 10 includes a plurality of light-emitting elements 11. The plurality of light-emitting elements 11 emit light in different wavelength ranges. The arrangement of the plurality of light-emitting elements 11 is appropriately set. The light-emitting element unit 10 shown in FIG. 2 includes three light-emitting elements 11. The number of light-emitting elements 11 is not limited to three. Two or more light-emitting elements 11 are provided in the light-emitting element unit 10. The light-emitting element unit 10 corresponds to an example of a light-emitting unit.

The light-emitting element 11 is implemented with a bare chip type or a shell type light emitting diode (LED). The light-emitting element 11 may be implemented with a laser diode. A configuration of the light-emitting element 11 is appropriately set according to a wavelength range of emitted light.

The light-receiving element unit 20 receives various types of light emitted by the light-emitting element unit 10. The light-receiving element unit 20 includes a light-receiving element 21 that receives various types of light. The light-receiving element 21 receives transmitted light or reflected light of light emitted by the light-emitting element unit 10. The transmitted light is light transmitted through the user M. The reflected light is light reflected inside the user M and transmitted through the inside of the user M. The light-receiving element 21 includes one or a plurality of photodiodes. The light-receiving element unit 20 corresponds to an example of a light-receiving unit.

### First Embodiment

A first embodiment shows a first measurement device 100a including two light-emitting elements 11. The first measurement device 100a is an example of the measurement device 100. The first embodiment shows an oxygen saturation concentration measurement method using the first measurement device 100a. The oxygen saturation concentration measurement method corresponds to an example of a biological information measurement method.

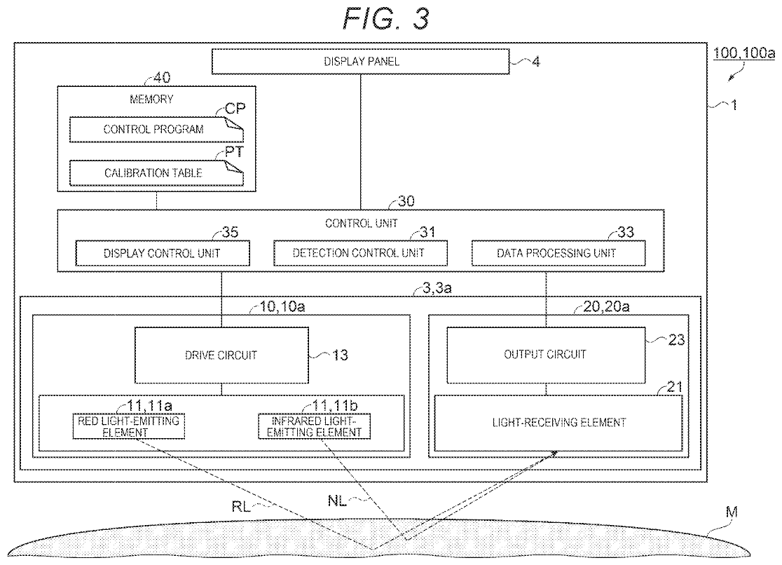

FIG. 3 shows a block configuration of the first measurement device 100a. FIG. 3 shows the first measurement device 100a excluding the belt 2. The first measurement device 100a accommodates various units or the like in the housing 1. The first measurement device 100a includes a first detection unit 3a, the control unit 30, the memory 40, and the display panel 4. The first detection unit 3a is an example of the detection unit 3.

The first detection unit 3a is an optical sensor module that detects, as a detection signal, data related to biological information measured using light in various wavelength ranges. The first detection unit 3a includes a first light-emitting element unit 10a and a first light-receiving element unit 20a. The first light-emitting element unit 10a is an example of the light-emitting element unit 10. The first light-receiving element unit 20a is an example of the light-receiving element unit 20.

The first light-emitting element unit 10a includes the plurality of light-emitting elements 11 and a drive circuit 13. The plurality of light-emitting elements 11 shown in FIG. 3 are a red light-emitting element 11a and an infrared light-emitting element 11b.

The red light-emitting element 11a emits red light RL toward the measurement site of the user M. The red light-emitting element 11a emits the red light RL in a wavelength range of 600 nm to 800 nm toward the measurement site. The red light RL is, for example, light having a peak wavelength of 660 nm. The red light-emitting element 11a corresponds to an example of a first light-emitting element.

The infrared light-emitting element 11b emits infrared light NL toward the measurement site of the user M. The infrared light-emitting element 11b emits the infrared light NL in a wavelength range of 800 nm to 1300 nm toward the measurement site. The infrared light NL is, for example, near infrared light having a peak wavelength of 905 nm. The infrared light-emitting element 11b corresponds to an example of a second light-emitting element.

The drive circuit 13 drives the plurality of light-emitting elements 11. The drive circuit 13 causes the plurality of light-emitting elements 11 to emit light under the control of the control unit 30. The drive circuit 13 causes the red light-emitting element 11a and the infrared light-emitting element 11b to emit light.

The light-receiving element unit 20 includes the light-receiving element 21 and an output circuit 23. The light-receiving element 21 receives reflected light emitted by the light-emitting element 11 and reflected by the measurement site of the user M. The light-receiving element 21 receives the red light RL and the infrared light NL reflected by the measurement site of the user M. The light-receiving element 21 alternately receives the red light RL and the infrared light NL in a time-division manner. The light-receiving element 21 may be divided into two regions. Each of the two regions receives either the red light RL or the infrared light NL. The light-receiving element 21 may be divided into a plurality of regions using an optical filter (not shown). The light-receiving element 21 receives at least one of the red light RL and the infrared light NL through the optical filter.

The light-receiving element unit 20 shown in FIG. 3 receives reflected light of the red light RL and reflected light of the infrared light NL, but is not limited thereto. The light-receiving element unit 20 may receive the red light RL transmitted through the user M and the infrared light NL transmitted through the user M. The light-receiving element unit 20 receives transmitted light of the red light RL and transmitted light of the infrared light NL.

The output circuit 23 outputs, to the control unit 30, a detection signal based on the light received by the light-receiving element 21. The output circuit 23 generates a detection signal by performing processing such as analog-to-digital conversion on light-receiving intensity data of the light received by the light-receiving element 21. The output circuit 23 generates a red light detection signal based on the red light RL received by the light-receiving element 21. The output circuit 23 generates an infrared light detection signal based on the infrared light NL received by the light-receiving element 21. The red light detection signal corresponds to an example of a first light-receiving signal. The infrared light detection signal corresponds to an example of a second light-receiving signal.

The control unit 30 is a controller that controls operations of various units. The control unit 30 is, for example, a processor including a central processing unit (CPU). The control unit 30 may include one or a plurality of processors. The control unit 30 may include a semiconductor memory such as a RAM (random access memory) or a read only memory (ROM). The semiconductor memory functions as a work area of the control unit 30. The control unit 30 functions as a detection control unit 31, a data processing unit 33, and a display control unit 35 by executing a control program CP stored in the memory 40. The control unit 30 corresponds to an example of a controller.

The detection control unit 31 is a functional unit that operates in the control unit 30. The detection control unit 31 controls the light-emitting element unit 10 and the light-receiving element unit 20. The detection control unit 31 adjusts a light-emitting timing, a light extinction timing, a light amount, or the like of the light-emitting element 11 via the drive circuit 13. The detection control unit 31 controls a light-receiving timing, a light-receiving time, digital-to-analog conversion, or the like of various types of light for the light-receiving element unit 20.

The data processing unit 33 is a functional unit that operates in the control unit 30. The data processing unit 33 processes the detection signal output from the light-receiving element unit 20. The data processing unit 33 acquires the red light detection signal and the infrared light detection signal from the light-receiving element unit 20.

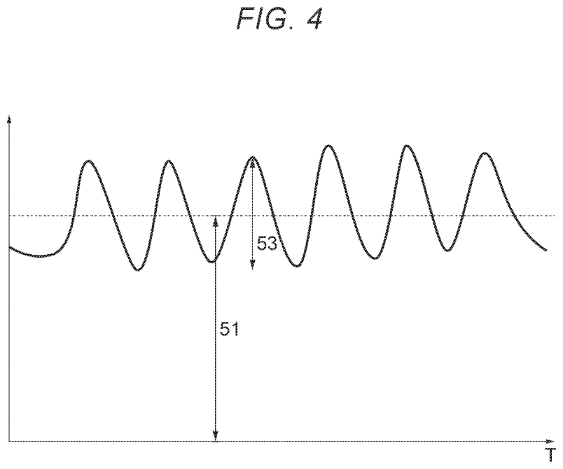

The data processing unit 33 calculates a DC component and an AC component from the detection signal. FIG. 4 schematically shows a detection signal. A horizontal axis in FIG. 4 indicates time. A vertical axis in FIG. 4 indicates an intensity of the detection signal. FIG. 4 schematically shows an example of the detection signal output from the output circuit 23.

The detection signal is data of a signal intensity detected at a predetermined interval. The signal intensity is detected n times per second. n is an integer of 1 or more. For example, n is 16. The signal intensity includes DC component data 51 as a DC component and AC component data 53 as an AC component. The data processing unit 33 separates the DC component data 51 and the AC component data 53 from the signal intensity. The data processing unit 33 calculates the AC component data 53 by performing time-frequency analysis.

The data processing unit 33 performs time-frequency analysis such as short time Fourier transform on the detection signal. The data processing unit 33 analyzes frequency information by performing the short time Fourier transform on the detection signal. The data processing unit 33 obtains a spectrogram in a predetermined frequency range by performing the short time Fourier transform on the detection signal. The predetermined frequency range is a range including the frequency of the pulse wave. The predetermined frequency range is, for example, a range of 0.5 Hz to 2 Hz. The predetermined frequency range is appropriately adjusted according to the wavelength range of the light subjected to the short time Fourier transform. The data processing unit 33 performs the short time Fourier transform on the red light detection signal to obtain a red light spectrogram. The data processing unit 33 performs the short time Fourier transform on the infrared light detection signal to obtain an infrared light spectrogram. The data processing unit 33 corresponds to an example of a controller.

The time-frequency analysis executed by the data processing unit 33 is not limited to the short time Fourier transform. The method is not limited as long as the frequency information for the detection signal can be analyzed. The data processing unit 33 may perform, for example, wavelet conversion.

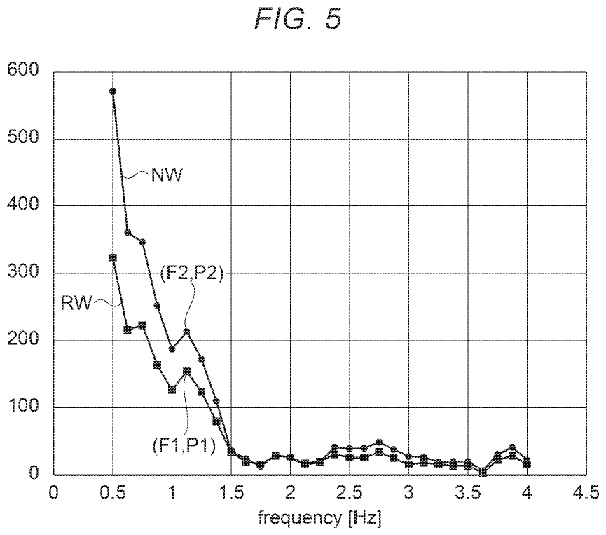

FIG. 5 shows a relationship between a frequency and a signal intensity of each detection signal in a predetermined time. FIG. 5 shows a part of a result of the short time Fourier transform. FIG. 5 shows red light data RW and infrared light data NW in the predetermined time. The red light data RW shown in FIG. 5 indicates a relationship between a frequency and a signal intensity of the red light RL at the predetermined time. The infrared light data NW shown in FIG. 5 indicates a relationship between a frequency and a signal intensity of the infrared light NL at the predetermined time.

The red light data RW indicates a first peak value P1 at a first frequency F1. The first frequency F1 corresponds to a frequency of a pulse wave. The data processing unit 33 acquires the signal intensity at each time at the first frequency F1 as a red light detection signal intensity.

The infrared light data NW indicates a second peak value P2 at a second frequency F2. The second frequency F2 is equal to or approximate to the first frequency F1. The second frequency F2 corresponds to a frequency of a pulse wave. The data processing unit 33 acquires the signal intensity at each time at the second frequency F2 as an infrared light detection signal intensity.

The data processing unit 33 acquires the red light detection signal intensity and the infrared light detection signal intensity at each time. The data processing unit 33 calculates a fluctuation component amplitude ratio using the red light detection signal intensity and the infrared light detection signal intensity. The fluctuation component amplitude ratio is a ratio between the red light transmitted amount and the infrared light transmitted amount. The red light transmitted amount is a light amount of the red light RL, which is emitted from the red light-emitting element 11a, is transmitted through the measurement site of the user M, and reaches the light-receiving element 21. The infrared light transmitted amount is a light amount of the infrared light NL, which is emitted from the infrared light-emitting element 11b, is transmitted through the measurement site of the user M, and reaches the light-receiving element 21. The fluctuation component amplitude ratio is calculated by the following formula (1).

R=(AC_{Red}/DC_{Red})/(AC_{IR}/DC_{IR}) (1)

Here, R indicates the fluctuation component amplitude ratio. AC_{Red} indicates an intensity of an AC component of the red light detection signal. DC_{Red} indicates an intensity of a DC component of the red light detection signal. AC_{IR} indicates an intensity of an AC component of the infrared light detection signal. DC_{IR} indicates an intensity of a DC component of the infrared light detection signal.

The intensity of the AC component of the red light detection signal is the red light detection signal intensity. The intensity of the DC component of the red light detection signal is a DC component separated from the red light detection signal at the predetermined time. The intensity of the AC component of the infrared light detection signal is the infrared light detection signal intensity. The intensity of the DC component of the infrared light detection signal is a DC component separated from the infrared light detection signal at the predetermined time.

The data processing unit 33 calculates the oxygen saturation concentration based on the calculated fluctuation component amplitude ratio. The data processing unit 33 refers to a calibration table PT stored in the memory 40 to obtain a value of the oxygen saturation concentration corresponding to the fluctuation component amplitude ratio. The data processing unit 33 determines, as the oxygen saturation concentration, the value of the oxygen saturation concentration corresponding to the fluctuation component amplitude ratio. The data processing unit 33 calculates the oxygen saturation concentration using the red light detection signal and the infrared light detection signal.

The data processing unit 33 calculates a correlation coefficient using the red light detection signal and the infrared light detection signal. The data processing unit 33 calculates a correlation coefficient between red light detection signal data RD and infrared light detection signal data ND.

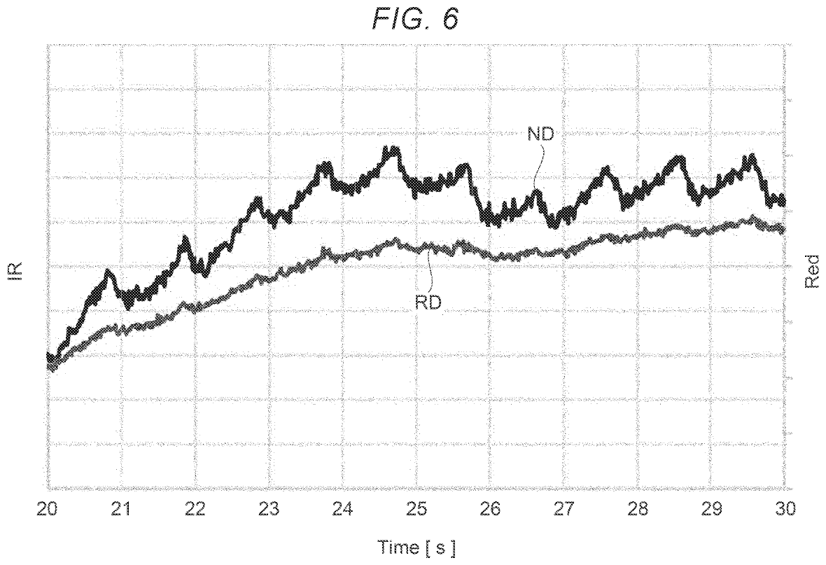

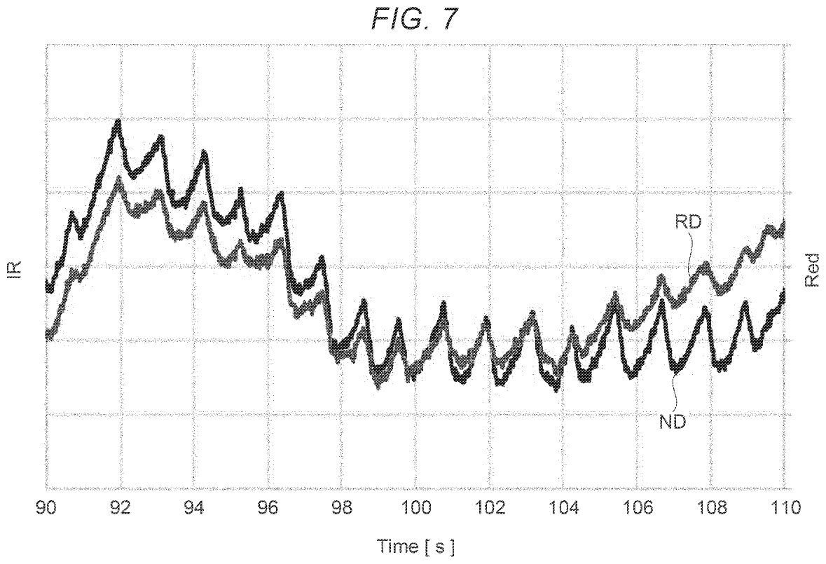

FIGS. 6 and 7 show the red light detection signal data RD and the infrared light detection signal data ND. The red light detection signal data RD shows a change with time of the red light detection signal. The infrared light detection signal data ND shows a change with time of the infrared light detection signal. FIGS. 6 and 7 show the red light detection signal data RD and the infrared light detection signal data ND in different time ranges. Horizontal axes in FIGS. 6 and 7 indicate measurement time. Left vertical axes in FIGS. 6 and 7 indicate signal values of the infrared light detection signal. Right vertical axes in FIGS. 6 and 7 indicate signal values of the red light detection signal.

FIG. 6 shows the red light detection signal data RD and the infrared light detection signal data ND when the measurement time is from 20 seconds to 30 seconds. The infrared light detection signal data ND can detect an AC component indicating the frequency of the pulse wave. On the other hand, it is difficult to detect the AC component in the red light detection signal data RD. The red light detection signal data RD is data in which it is difficult to detect the AC component due to an influence of body movement of the user M, an environmental temperature, or the like. The red light detection signal data RD and the infrared light detection signal data ND are represented by curves with low approximation. When at least one AC component of the red light detection signal data RD and the infrared light detection signal data ND is difficult to detect, the measurement accuracy of the calculated oxygen saturation concentration decreases.

FIG. 7 shows the red light detection signal data RD and the infrared light detection signal data ND when the measurement time is from 90 seconds to 110 seconds. The infrared light detection signal data ND can detect an AC component indicating the frequency of the pulse wave. On the other hand, unlike the red light detection signal data RD shown in FIG. 6, the red light detection signal data RD can detect the AC component. The red light detection signal data RD and the infrared light detection signal data ND easily detect the AC component. The red light detection signal data RD and the infrared light detection signal data ND are represented by curves with high approximation. When the approximation between the red light detection signal data RD and the infrared light detection signal data ND is high, the measurement accuracy of the calculated oxygen saturation concentration is improved. The approximation between the red light detection signal data RD and the infrared light detection signal data ND corresponds to the measurement accuracy of the oxygen saturation concentration. The data processing unit 33 can evaluate the reliability of the calculated oxygen saturation concentration by calculating the correlation coefficient indicating the approximation between the red light detection signal data RD and the infrared light detection signal data ND. The correlation coefficient corresponds to an example of correlation data. The data processing unit 33 calculates the correlation coefficient using a correlation coefficient calculation formula represented by the following formula (2).

$$r = \frac{{\sum }_{n = 1}^{N} ( {x}_{n} - {x}_{a v e} ) \{ {y}_{n} - {y}_{a v e} )}{\sqrt{{\sum }_{n = 1}^{N} {( {x}_{n} - {x}_{ave} )}^{2} {\sum }_{n = 1}^{N} {( {y}_{n} - {y}_{ave} )}^{2}}} \qquad ( 2 )$$

Here, r represents a correlation coefficient. N represents the number of red light detection signals used for calculating the correlation coefficient. xₙ indicates a red light detection signal at each measurement time. n is an integer of 1 or more. xₐᵥₑ indicates an average value of the red light detection signals within a predetermined time. yₙ indicates an infrared light detection signal at each measurement time. yₐᵥₑ indicates an average value of the infrared light detection signals within the predetermined time. The predetermined time is, for example, 8 seconds. When the red light detection signal and the infrared light detection signal are measured k times per second, N is 8×k. k is an integer of 1 or more.

The data processing unit 33 shown in FIG. 3 calculates the correlation coefficient in units of predetermined time intervals. For example, the data processing unit 33 calculates the correlation coefficient for the measurement time at 8 seconds using the detection signal for the measurement time of 1 second to 8 seconds. The data processing unit 33 calculates the correlation coefficient for the measurement time at 9 seconds using the detection signal for the measurement time of 2 seconds to 9 seconds. The data processing unit 33 calculates a correlation coefficient of each measurement time. The predetermined time interval is not limited to 8 seconds. The predetermined time interval is appropriately set in advance. The data processing unit 33 calculates the correlation coefficient at a timing of calculating the oxygen saturation concentration. The data processing unit 33 calculates the correlation coefficient and the oxygen saturation concentration at the predetermined time interval.

The data processing unit 33 may set a predetermined time interval by referring to at least one of the red light detection signal data RD and the infrared light detection signal data ND. The data processing unit 33 sets a time range including at least one pulse wave as the predetermined time interval. The frequency of the pulse wave varies depending on the user M. By referring to at least one of the red light detection signal data RD and the infrared light detection signal data ND, the data processing unit 33 can include pulse waves in data for calculating the correlation coefficient.

The data processing unit 33 determines the oxygen saturation concentration based on the calculated correlation coefficient. As the correlation coefficient is closer to 1, the measurement accuracy of the oxygen saturation concentration is higher. The higher the measurement accuracy of the oxygen saturation concentration, the higher the reliability of the calculated oxygen saturation concentration. The data processing unit 33 can evaluate the reliability of the oxygen saturation concentration calculated using the correlation coefficient.

The data processing unit 33 may determine the oxygen saturation concentration by comparing the calculated correlation coefficient with a correlation coefficient threshold. The correlation coefficient threshold is stored in the memory 40 in advance. The correlation coefficient threshold is used to evaluate the calculated correlation coefficient. The data processing unit 33 reads the correlation coefficient threshold from the memory 40 and compares the correlation coefficient threshold with the correlation coefficient. For example, when the correlation coefficient is larger than the correlation coefficient threshold, the data processing unit 33 determines that the reliability of the oxygen saturation concentration is high. The correlation coefficient threshold corresponds to an example of a threshold.

A plurality of correlation coefficient thresholds may be stored in the memory 40 in advance. The data processing unit 33 may determine the oxygen saturation concentration by comparing the calculated correlation coefficient with the plurality of correlation coefficient thresholds.

The data processing unit 33 outputs the oxygen saturation concentration to the display control unit 35. The data processing unit 33 may output the oxygen saturation concentration to an external device via a communication interface (not shown). The data processing unit 33 may determine whether to output the oxygen saturation concentration to the display control unit 35 based on a result obtained by comparing the correlation coefficient with the correlation coefficient threshold. When the calculated correlation coefficient is larger than the correlation coefficient threshold, the data processing unit 33 outputs the oxygen saturation concentration to the display control unit 35. When the calculated correlation coefficient is smaller than the correlation coefficient threshold, the data processing unit 33 deletes the oxygen saturation concentration and does not output the oxygen saturation concentration to the display control unit 35. The data processing unit 33 does not output the oxygen saturation concentration with low reliability. For example, the oxygen saturation concentration with low reliability is not displayed on the display panel 4.

The data processing unit 33 may calculate reliability data of the oxygen saturation concentration based on the calculated correlation coefficient. For example, the data processing unit 33 calculates the reliability data using a conversion formula for converting the correlation coefficient into the reliability data. The conversion formula is appropriately set in advance by a manufacturer of the measurement device 100. The data processing unit 33 may calculate the reliability data with reference to a conversion table (not shown). The conversion table is a table that associates the correlation coefficient with the reliability data. The conversion table is stored in the memory 40 in advance. The data processing unit 33 outputs the calculated reliability data to the display control unit 35. The reliability data corresponds to the measurement accuracy of the oxygen saturation concentration. The reliability data corresponds to an example of reliability.

The display control unit 35 is a functional unit that operates in the control unit 30. The display control unit 35 controls display of the display panel 4. The display control unit 35 causes the display panel 4 to display various images by transmitting display data to the display panel 4.

The display control unit 35 acquires the oxygen saturation concentration from the data processing unit 33 at a predetermined timing. The display control unit 35 generates the display data including the oxygen saturation concentration. The display control unit 35 outputs the display data including the oxygen saturation concentration to the display panel 4. The display control unit 35 causes the display panel 4 to display the oxygen saturation concentration based on the display data. The display control unit 35 may cause the display panel 4 to display a moving average of the oxygen saturation concentrations based on the display data including the moving average of the oxygen saturation concentrations. The display control unit 35 corresponds to an example of a controller.

When the oxygen saturation concentration is not output from the data processing unit 33 at the predetermined timing, the display control unit 35 does not display the oxygen saturation concentration. The display control unit 35 causes the display panel 4 to continuously display the oxygen saturation concentration output before the timing at which the oxygen saturation concentration is not output. The display control unit 35 may cause the display panel 4 to display an image indicating that an oxygen saturation concentration with a predetermined measurement accuracy is not measured.

The display control unit 35 acquires reliability data from the data processing unit 33 at a predetermined timing. The display control unit 35 generates display data including the reliability data. The display control unit 35 may generate display data including the oxygen saturation concentration and the reliability data. The display control unit 35 outputs the display data including the reliability data to the display panel 4. The display control unit 35 causes the display panel 4 to display the reliability data based on the display data. The display control unit 35 may cause the display panel 4 to simultaneously display the oxygen saturation concentration and the reliability data. The display control unit 35 may cause the display panel 4 to display the reliability data after causing the display panel 4 to display the oxygen saturation concentration. The display control unit 35 may cause the display panel 4 to display the reliability data when the user M performs a predetermined operation on the measurement device 100.

For example, the display control unit 35 causes the display panel 4 to display the reliability data in percentage. The display control unit 35 causes the display panel 4 to display the reliability data with data reliability of 80%, 90%, 95%, or the like. The reliability data is obtained by converting the correlation coefficient using conversion data. The display panel 4 displays the reliability data in the form of data reliability.

The memory 40 stores various types of data. The memory 40 stores control data for operating various units, various types of data calculated by the control unit 30, or the like. The memory 40 may store the oxygen saturation concentration or the like calculated by the data processing unit 33. The memory 40 stores the control program CP that operates in the control unit 30. The memory 40 stores the correlation coefficient calculation formula. The memory 40 stores the calibration table PT referred to by the data processing unit 33. The memory 40 may store the conversion formula or the conversion table. The memory 40 includes a ROM, a RAM, or the like. The memory 40 corresponds to an example of a storage unit.

The control program CP is executed by the control unit 30 to operate various functional units. The control program CP causes the control unit 30 to operate as the detection control unit 31, the data processing unit 33, and the display control unit 35. The control program CP may cause the control unit 30 to operate as a functional unit other than the detection control unit 31, the data processing unit 33, and the display control unit 35.

The calibration table PT is a table that stores the fluctuation component amplitude ratio and the oxygen saturation concentration in association with each other. The calibration table PT shows a relationship between the fluctuation component amplitude ratio and the oxygen saturation concentration. The calibration table PT is created in advance by the manufacturer of the measurement device 100. The data processing unit 33 determines the oxygen saturation concentration corresponding to the calculated fluctuation component amplitude ratio by referring to the calibration table PT. The calibration table PT corresponds to an example of a calibration curve table.

The memory 40 may store a calibration formula instead of the calibration table PT. The calibration formula is a relational expression between the fluctuation component amplitude ratio and the oxygen saturation concentration. The data processing unit 33 calculates the oxygen saturation concentration corresponding to the calculated fluctuation component amplitude ratio using the calibration formula.

The display panel 4 displays various images. The display panel 4 displays the oxygen saturation concentration under the control of the display control unit 35. The display panel 4 may display the reliability data under the control of the display control unit 35. The display panel 4 displays the oxygen saturation concentration based on the display data output from the display control unit 35. The display panel 4 displays the reliability data based on the display data output from the display control unit 35. The display panel 4 may display a pulse rate or the like. The display panel 4 includes a liquid crystal display, an organic electro-luminescence (EL) display, or the like.

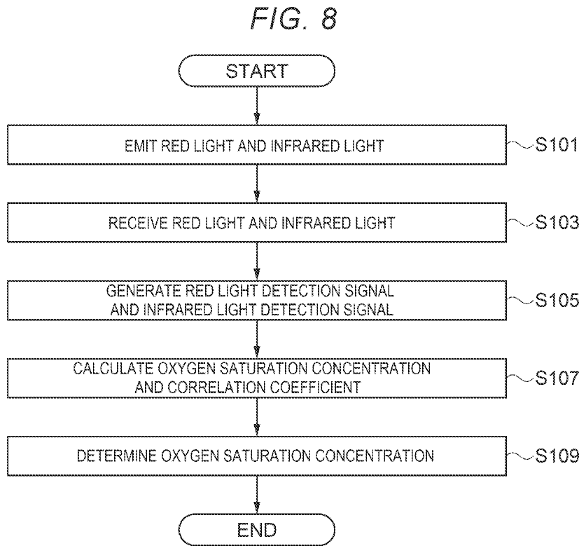

FIG. 8 shows a flowchart for determining an oxygen saturation concentration. The flowchart shown in FIG. 8 shows an oxygen saturation concentration measurement method. The oxygen saturation concentration measurement method corresponds to an example of a biological information measurement method. FIG. 8 shows an oxygen saturation concentration measurement method executed by the first measurement device 100a.

In step S101, the first measurement device 100a emits the red light RL and the infrared light NL. The detection control unit 31 operated in the control unit 30 causes, via the drive circuit 13, the light-emitting element 11 to emit light. The detection control unit 31 controls the light-emitting element unit 10 to emit light to the user M. The detection control unit 31 causes the light-emitting element unit 10 to emit the red light RL to the user M. The red light-emitting element 11a emits the red light RL to the user M. The detection control unit 31 causes the light-emitting element unit 10 to emit the infrared light NL to the user M. The infrared light-emitting element 11b emits the infrared light NL to the user M.

After emitting the red light RL and the infrared light NL, the first measurement device 100a receives the red light RL and the infrared light NL in step S103. The detection control unit 31 causes the light-receiving element unit 20 to receive the red light RL and the infrared light NL that passed through the user M. The light-receiving element 21 of the light-receiving element unit 20 receives the red light RL and the infrared light NL reflected by the user M. The light-receiving element 21 receives the red light RL and the infrared light NL at different timings. The light-receiving element 21 may receive the red light RL and the infrared light NL in different regions.

After receiving the red light RL and the infrared light NL, the first measurement device 100a generates the red light detection signal and the infrared light detection signal in step S105. The output circuit 23 of the light-receiving element unit 20 generates the red light detection signal based on the received red light RL. The output circuit 23 generates the red light detection signal by performing processing such as analog-to-digital conversion on the light-receiving intensity data of the red light RL detected by the light-receiving element 21. The output circuit 23 generates the infrared light detection signal based on the received infrared light NL. The output circuit 23 generates the infrared light detection signal by performing processing such as analog-to-digital conversion on the light-receiving intensity data of the infrared light NL detected by the light-receiving element 21.

After generating the red light detection signal and the infrared light detection signal, the first measurement device 100a calculates the oxygen saturation concentration and the correlation coefficient in step S107. The data processing unit 33 calculates the oxygen saturation concentration using the red light detection signal and the infrared light detection signal. The data processing unit 33 calculates the correlation coefficient based on the red light detection signal and the infrared light detection signal.

For example, the data processing unit 33 performs the short time Fourier transform on the red light detection signal and the infrared light detection signal. The data processing unit 33 detects the first frequency F1 corresponding to the frequency of the pulse wave by performing the short time Fourier transform on the red light detection signal. The data processing unit 33 acquires the first peak value P1, which is a signal intensity of the first frequency F1, as the red light detection signal intensity. The data processing unit 33 detects the second frequency F2 corresponding to the frequency of the pulse wave by performing the short time Fourier transform on the infrared light detection signal. The data processing unit 33 acquires the second peak value P2, which is a signal intensity of the second frequency F2, as the infrared light detection signal intensity.

The data processing unit 33 acquires the red light detection signal intensity and the infrared light detection signal intensity at predetermined time intervals. The data processing unit 33 calculates a fluctuation component amplitude ratio using the red light detection signal intensity and the infrared light detection signal intensity. The data processing unit 33 calculates the fluctuation component amplitude ratio using Formula (1).

The data processing unit 33 calculates the oxygen saturation concentration based on the calculated fluctuation component amplitude ratio. The data processing unit 33 refers to the calibration table PT stored in the memory 40 to obtain the oxygen saturation concentration corresponding to the fluctuation component amplitude ratio.

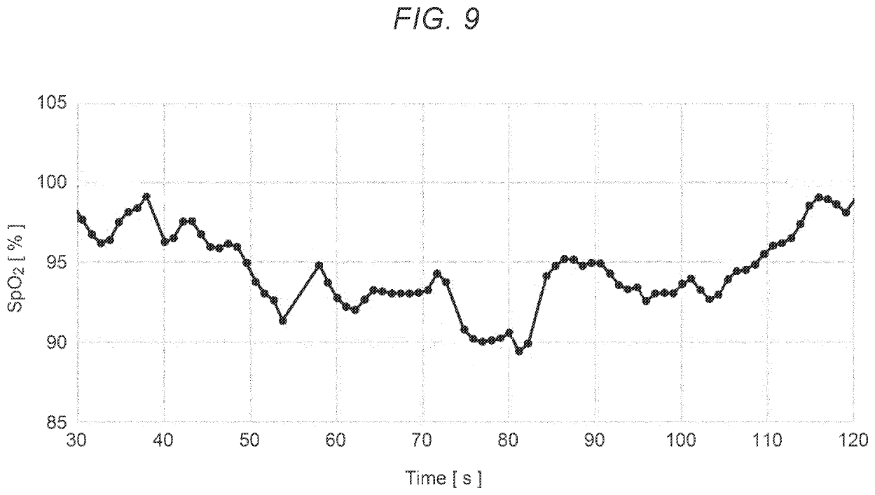

FIG. 9 shows a measurement result of the oxygen saturation concentration. FIG. 9 shows a change with time of the oxygen saturation concentration. FIG. 9 is a diagram plotting a change in the oxygen saturation concentration displayed on the display panel 4. The oxygen saturation concentration fluctuates depending on the measurement time. The oxygen saturation concentration fluctuates depending on the body movement or the like of the user M. The oxygen saturation concentration shown in FIG. 9 includes data with a low measurement accuracy.

The data processing unit 33 calculates the correlation coefficient based on the red light detection signal and the infrared light detection signal. The data processing unit 33 calculates the correlation coefficient in units of predetermined time intervals. The data processing unit 33 calculates the correlation coefficient using the red light detection signal intensity and the infrared light detection signal intensity at each time. The data processing unit 33 calculates the correlation coefficient using a correlation coefficient calculation formula represented by Formula (2).

After calculating the oxygen saturation concentration and the correlation coefficient, the first measurement device 100a determines the oxygen saturation concentration in step S109. The data processing unit 33 determines the oxygen saturation concentration based on the correlation coefficient. The data processing unit 33 determines that the measurement accuracy of the oxygen saturation concentration is higher as the correlation coefficient is closer to 1. The first measurement device 100a can determine the measurement accuracy of the oxygen saturation concentration by determining the oxygen saturation concentration.

The data processing unit 33 may determine the oxygen saturation concentration by comparing the calculated correlation coefficient with the correlation coefficient threshold. For example, when the calculated correlation coefficient is lower than the correlation coefficient threshold, the data processing unit 33 does not output the oxygen saturation concentration to the display control unit 35. The display control unit 35 does not cause the display panel 4 to display the oxygen saturation concentration having a correlation coefficient lower than the correlation coefficient threshold.

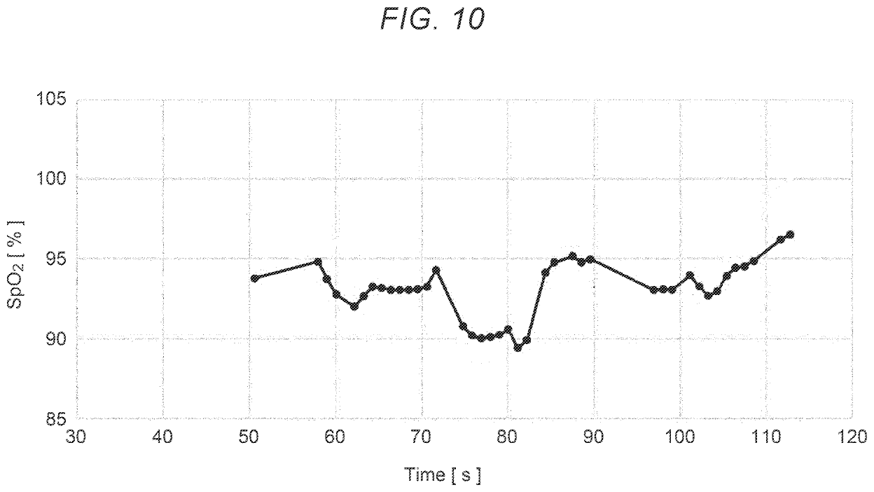

FIG. 10 shows a measurement result of the oxygen saturation concentration. FIG. 10 shows the output oxygen saturation concentration determined by the correlation coefficient. FIG. 10 does not show the oxygen saturation concentration in the measurement times from 30 seconds to 50 seconds and from 115 seconds to 120 seconds. The data processing unit 33 compares the calculated correlation coefficient with the correlation coefficient threshold, and does not output, to the display control unit 35, the oxygen saturation concentration in a range in which the correlation coefficient is lower than the correlation coefficient threshold. When the data processing unit 33 does not output the oxygen saturation concentration, the display control unit 35 does not cause the display panel 4 to display the oxygen saturation concentration.

For example, FIG. 10 shows a measurement result when the correlation coefficient threshold is 0.925. The correlation coefficient in the measurement time from 50 seconds to 110 seconds indicates a value of 0.925 or more. On the other hand, the correlation coefficient in the measurement time from 30 seconds to 50 seconds and the correlation coefficient in the measurement time from 115 seconds to 120 seconds indicate values less than 0.925. The correlation coefficient in the measurement time of 50 seconds is a correlation coefficient between the red light detection signal intensity and the infrared light detection signal intensity at a time interval in the measurement time from 42 seconds to 50 seconds. The correlation coefficient in each measurement time is a correlation coefficient at a time interval in units of 8 seconds.

The first measurement device 100a includes: the first light-emitting element unit 10a including the red light-emitting element 11a that emits the red light RL and the infrared light-emitting element 11b that emits the infrared light NL; the first light-receiving element unit 20a that receives the red light RL emitted from the red light-emitting element 11a and the infrared light NL emitted from the infrared light-emitting element 11b and generates the red light detection signal based on the red light RL and the infrared light detection signal based on the infrared light NL; and the control unit 30 that calculates the oxygen saturation concentration. The control unit 30 calculates the oxygen saturation concentration using the red light detection signal and the infrared light detection signal, calculates the correlation coefficient using the red light detection signal and the infrared light detection signal, and determines the oxygen saturation concentration based on the correlation coefficient.

The correlation coefficient fluctuates depending on the body movement or the like of the user M. When the correlation coefficient decreases, the measurement accuracy of the calculated oxygen saturation concentration decreases. The first measurement device 100a can determine the measurement accuracy of the calculated oxygen saturation concentration based on the calculated correlation coefficient.

The first measurement device 100a includes the memory 40 that stores the correlation coefficient threshold for evaluating the correlation coefficient. The control unit 30 determines the oxygen saturation concentration by comparing the calculated correlation coefficient with the correlation coefficient threshold.

By comparing the correlation coefficient threshold with the calculated correlation coefficient, the first measurement device 100a can easily determine the measurement accuracy of the oxygen saturation concentration.

The control unit 30 outputs the oxygen saturation concentration when the correlation coefficient is larger than the correlation coefficient threshold, and deletes the oxygen saturation concentration when the correlation coefficient is smaller than the correlation coefficient threshold.

The oxygen saturation concentration with a predetermined measurement accuracy is output. The oxygen saturation concentration with a low measurement accuracy is deleted and is not displayed to the user M.

The first measurement device 100a includes the display panel 4 that displays the oxygen saturation concentration. The control unit 30 calculates the reliability data of the oxygen saturation concentration based on the correlation coefficient, and causes the display panel 4 to display the reliability data.

The reliability data of the oxygen saturation concentration is displayed. The user M can grasp the measurement accuracy of the oxygen saturation concentration by checking the reliability data.

The control unit 30 calculates the correlation coefficient and the oxygen saturation concentration at the predetermined time interval.

The first measurement device 100a can estimate the measurement accuracy of the oxygen saturation concentration calculated at the predetermined time interval. The user M can check the change with time of the oxygen saturation concentration.

The control unit 30 calculates the frequency of the pulse wave based on the red light detection signal or the infrared light detection signal, and determines the predetermined time interval including one or more pulse waves using the frequency.

The frequency of the pulse wave fluctuates depending on the user M. When the user M is different, the frequency can be adjusted to a time interval including a pulse wave component.

The oxygen saturation concentration measurement method includes: emitting the red light RL and the infrared light NL to the user M; receiving the red light RL and the infrared light NL passing through the user M; generating the red light detection signal based on the received red light RL and the infrared light detection signal based on the received infrared light NL; calculating the oxygen saturation concentration using the red light detection signal and the infrared light detection signal; calculating the correlation coefficient based on the red light detection signal and the infrared light detection signal; and determining the oxygen saturation concentration based on the correlation coefficient.

The measurement accuracy of the calculated oxygen saturation concentration is determined. The first measurement device 100a can check the measurement accuracy of the calculated oxygen saturation concentration.

### Second Embodiment

A second embodiment shows a second measurement device 100b including three light-emitting elements 11. The second measurement device 100b is an example of the measurement device 100. The second embodiment shows an oxygen saturation concentration measurement method using the second measurement device 100b.

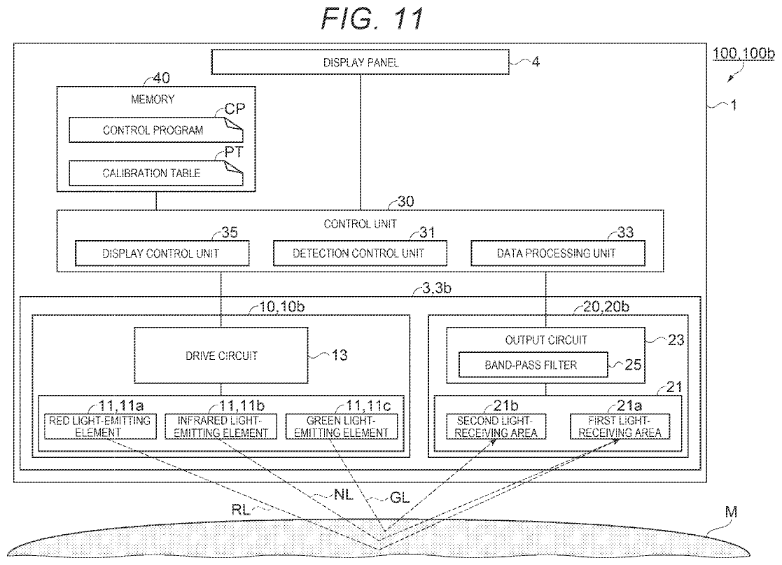

FIG. 11 shows a block configuration of the measurement device 100. FIG. 11 shows the second measurement device 100b excluding the belt 2. The second measurement device 100b accommodates various units or the like in the housing 1. The second measurement device 100b includes a second detection unit 3b, the control unit 30, the memory 40, and the display panel 4. The second detection unit 3b includes a second light-emitting element unit 10b and a second light-receiving element unit 20b. The second detection unit 3b is an example of the detection unit 3. A configuration of the second measurement device 100b is the same as the configuration of the first measurement device 100a except for the detection unit 3. Hereinafter, configurations and functions of the second detection unit 3b different from those of the first measurement device 100a will be described.

The second light-emitting element unit 10b provided in the second detection unit 3b includes three light-emitting elements 11 and the drive circuit 13. The second light-emitting element unit 10b is an example of the light-emitting element unit 10. The three light-emitting elements 11 are the red light-emitting element 11a, the infrared light-emitting element 11b, and a green light-emitting element 11c. The red light-emitting element 11a emits the red light RL toward the measurement site of the user M. The infrared light-emitting element 11b emits the infrared light NL toward the measurement site of the user M. The green light-emitting element 11c emits green light GL toward the measurement site of the user M. The green light-emitting element 11c corresponds to an example of a third light-emitting element.

The drive circuit 13 drives the three light-emitting elements 11. The drive circuit 13 causes the three light-emitting elements 11 to emit light under the control of the control unit 30. The drive circuit 13 causes the red light-emitting element 11a, the infrared light-emitting element 11b, and the green light-emitting element 11c to emit light.

The second light-receiving element unit 20b provided in the second detection unit 3b includes the light-receiving element 21 and the output circuit 23. The second light-receiving element unit 20b is an example of the light-receiving element unit 20. The light-receiving element 21 receives light emitted by the light-emitting element 11 and reflected by the measurement site of the user M. The light-receiving element 21 receives the red light RL, the infrared light NL, and the green light GL reflected by the measurement site of the user M. The light-receiving element 21 is divided into a plurality of regions. The light-receiving element 21 may be divided into the plurality of regions using an optical filter (not shown). The light-receiving element 21 shown in FIG. 11 is divided into a first light-receiving area 21a and a second light-receiving area 21b.

The first light-receiving area 21a receives the red light RL and the infrared light NL. The first light-receiving area 21a receives the red light RL emitted by the red light-emitting element 11a and reflected by the measurement site of the user M. The first light-receiving area 21a receives the infrared light NL emitted by the infrared light-emitting element 11b and reflected by the measurement site of the user M. The first light-receiving area 21a may receive at least one of the red light RL and the infrared light NL through the optical filter. The first light-receiving area 21a may alternately receive the red light RL and the infrared light NL in a time-division manner.

The second light-receiving area 21b receives the green light GL. The second light-receiving area 21b receives the green light GL emitted by the green light-emitting element 11c and reflected by the measurement site of the user M. The second light-receiving area 21b may receive the green light GL via the optical filter.

In FIG. 11, the red light RL and the infrared light NL are received by the first light-receiving area 21a, but are not limited thereto. A third light-receiving area different from the first light-receiving area 21a and the second light-receiving area 21b may be provided. The red light RL or the infrared light NL may be received by the third light-receiving area. At this time, when the third light-receiving area receives the infrared light NL, the first light-receiving area 21a receives the red light RL. The light-receiving element 21 may not be divided into the plurality of regions. The light-receiving element 21 may receive the red light RL, the infrared light NL, and the green light GL in a time-division manner.

The light-receiving element unit 20 shown in FIG. 11 receives reflected light of the red light RL and reflected light of the infrared light NL, but is not limited thereto. The light-receiving element unit 20 may receive the red light RL transmitted through the user M and the infrared light NL transmitted through the user M. The light-receiving element unit 20 receives transmitted light of the red light RL and transmitted light of the infrared light NL.

The output circuit 23 outputs, to the control unit 30, a detection signal based on the light received by the light-receiving element 21. The output circuit 23 generates a detection signal by performing processing such as analog-to-digital conversion on light-receiving intensity data of the light received by the light-receiving element 21. The output circuit 23 generates a red light detection signal based on the red light RL received by the first light-receiving area 21a. The output circuit 23 generates an infrared light detection signal based on the infrared light NL received by the first light-receiving area 21a. The output circuit 23 generates a green light detection signal based on the green light GL received by the second light-receiving area 21b. The red light detection signal corresponds to an example of a first light-receiving signal. The infrared light detection signal corresponds to an example of a second light-receiving signal. The green light detection signal corresponds to an example of a third light-receiving signal.

The output circuit 23 includes a band-pass filter 25. The band-pass filter 25 extracts an AC component from the light-receiving intensity data. The band-pass filter 25 separates the light-receiving intensity data into an AC component and a DC component by extracting the AC component from the light-receiving intensity data. The AC component corresponds to the AC component data 53 shown in FIG. 4. The DC component corresponds to the DC component data 51 shown in FIG. 4. The band-pass filter 25 outputs the separated AC component and the DC component as detection signals to the control unit 30. The band-pass filter 25 corresponds to an example of a filter. The AC component corresponds to an example of a fluctuation component.

The band-pass filter 25 extracts a red light AC component from the red light RL received by the first light-receiving area 21a. The band-pass filter 25 separates a red light AC component and a red light DC component by extracting the red light AC component. The red light AC component corresponds to an example of a first fluctuation component. The band-pass filter 25 extracts an infrared light AC component from the infrared light NL received by the first light-receiving area 21a. The band-pass filter 25 separates an infrared light AC component and an infrared light DC component by extracting the infrared light AC component. The infrared light AC component corresponds to an example of a second fluctuation component. The output circuit 23 outputs the red light AC component and the red light DC component as red light detection signals to the control unit 30. The output circuit 23 outputs the infrared light AC component and the infrared light DC component as infrared light detection signals to the control unit 30.

The band-pass filter 25 may extract a green light AC component from the green light GL received by the second light-receiving area 21b. The band-pass filter 25 separates a green light AC component and a green light DC component by extracting the green light AC component. The output circuit 23 outputs the green light AC component and the green light DC component as green light detection signals to the control unit 30.

The data processing unit 33 is a functional unit that operates in the control unit 30. The data processing unit 33 processes the detection signal output from the light-receiving element unit 20. The data processing unit 33 acquires the red light detection signal, the infrared light detection signal, and the green light detection signal from the light-receiving element unit 20.

The data processing unit 33 performs short time Fourier transform on the detection signal. The data processing unit 33 analyzes frequency information by performing the short time Fourier transform on the detection signal. The data processing unit 33 obtains a spectrogram in a predetermined frequency range by performing the short time Fourier transform on the detection signal. The predetermined frequency range is a range including the frequency of the pulse wave. The predetermined frequency range is, for example, a range of 0.5 Hz to 2 Hz. The predetermined frequency range is appropriately adjusted according to a wavelength range of light subjected to the short time Fourier transform. The data processing unit 33 performs the short time Fourier transform on the red light detection signal to obtain a red light spectrogram. The data processing unit 33 performs the short time Fourier transform on the infrared light detection signal to obtain an infrared light spectrogram. The data processing unit 33 performs the short time Fourier transform on the green light detection signal to obtain a green light spectrogram.

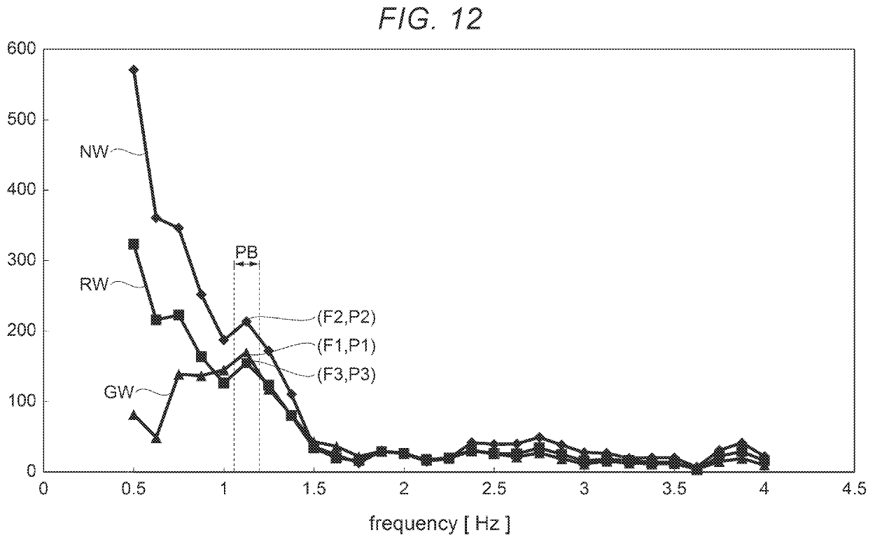

FIG. 12 shows a relationship between a frequency and a signal intensity of each detection signal in a predetermined time. The data processing unit 33 determines a pulse wave region PB shown in FIG. 12 using the green light spectrogram. The pulse wave region PB is a region including the frequency of the pulse wave. The pulse wave region PB is a frequency region including the frequency of the pulse wave for each time. The pulse wave region PB is, for example, a region including a third frequency F3 indicating a third peak value P3 in the green light detection signal. The pulse wave region PB corresponds to an example of a pulsation band. The green light detection signal is less susceptible to disturbance caused by body movement or the like than the red light detection signal and the infrared light detection signal. The data processing unit 33 can specify the frequency of the pulse wave with a high measurement accuracy by determining the pulse wave region PB using the green light spectrogram.

The data processing unit 33 detects a red light detection signal intensity of the pulse wave region PB and an infrared light detection signal intensity of the pulse wave region PB. The red light detection signal intensity represents a signal intensity of the red light detection signal in the pulse wave region PB. The red light detection signal intensity is, for example, the first peak value P1 of the red light detection signal in the pulse wave region PB. The infrared light detection signal intensity represents a signal intensity of the infrared light detection signal in the pulse wave region PB. The infrared light detection signal intensity is, for example, the second peak value P2 of the infrared light detection signal in the pulse wave region PB.

The data processing unit 33 calculates a fluctuation component amplitude ratio using the red light detection signal intensity and the infrared light detection signal intensity included in the pulse wave region PB. The data processing unit 33 calculates the fluctuation component amplitude ratio using Formula (1).

The data processing unit 33 determines an oxygen saturation concentration based on the calculated fluctuation component amplitude ratio. The data processing unit 33 refers to the calibration table PT stored in the memory 40 to obtain the oxygen saturation concentration corresponding to the calculated fluctuation component amplitude ratio. The data processing unit 33 outputs the oxygen saturation concentration to the display control unit 35. The data processing unit 33 may output the oxygen saturation concentration to an external device via a communication interface (not shown).

The data processing unit 33 may calculate the oxygen saturation concentration using the red light AC component and the infrared light AC component. The data processing unit 33 calculates the fluctuation component amplitude ratio using the red light AC component and the infrared light AC component. The data processing unit 33 refers to the calibration table PT to obtain the oxygen saturation concentration corresponding to the calculated fluctuation component amplitude ratio.

The data processing unit 33 calculates a correlation coefficient using the red light detection signal, the infrared light detection signal, and the green light detection signal. The data processing unit 33 determines the oxygen saturation concentration by calculating the correlation coefficient using the red light detection signal, the infrared light detection signal, and the green light detection signal.

For example, the data processing unit 33 calculates a correlation coefficient between the red light detection signal and the infrared light detection signal, a correlation coefficient between the red light detection signal and the green light detection signal, and a correlation coefficient between the infrared light detection signal and the green light detection signal. The correlation coefficient between the red light detection signal and the infrared light detection signal is represented as a first correlation coefficient. The correlation coefficient between the red light detection signal and the green light detection signal is represented as a second correlation coefficient. The correlation coefficient between the infrared light detection signal and the green light detection signal is represented as a third correlation coefficient.

The data processing unit 33 calculates an evaluation value for determining the oxygen saturation concentration using the first correlation coefficient, the second correlation coefficient, and the third correlation coefficient. The evaluation value corresponds to an example of correlation data. The data processing unit 33 may set a product of the first correlation coefficient, the second correlation coefficient, and the third correlation coefficient as the evaluation value. The data processing unit 33 may set a linear sum shown in Formula (3) as the evaluation value.

rₑ=a×r₁¹+b×r₂^{m}+c×r₃ⁿ+d (3)

Here, rₑ represents the evaluation value. r₁, r₂, and r₃ represent the first correlation coefficient, the second correlation coefficient, and the third correlation coefficient. a, b, c, l, m, and n are any constants. a, b, c, l, m, and n are appropriately set.

The data processing unit 33 may calculate the first correlation coefficient, the second correlation coefficient, and the third correlation coefficient using the red light AC component, the infrared light AC component, and the green light AC component. The data processing unit 33 calculates the evaluation value for determining the oxygen saturation concentration using the first correlation coefficient, the second correlation coefficient, and the third correlation coefficient.

The data processing unit 33 may output the calculated evaluation value as reliability data to the display control unit 35. The data processing unit 33 may obtain the reliability data corresponding to the evaluation value with reference to a conversion table that associates the evaluation value with the reliability data. The data processing unit 33 may calculate the reliability data by known machine learning of an algorithm using the first correlation coefficient, the second correlation coefficient, and the third correlation coefficient. A decision tree, a random forest, a support vector machine, a neural network, or the like is used for the machine learning.

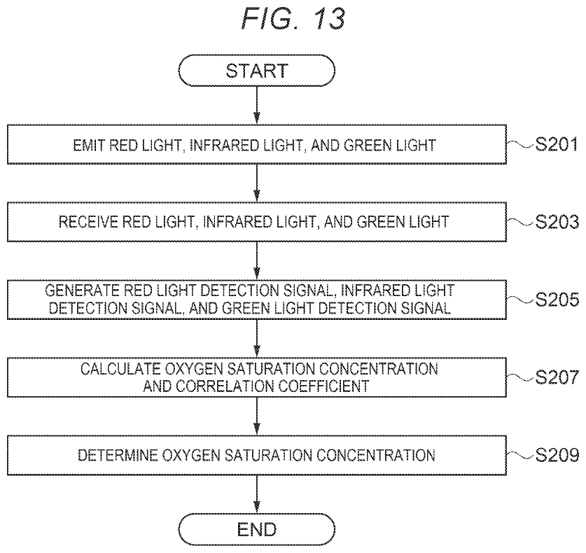

FIG. 13 shows a flowchart for determining the oxygen saturation concentration. The flowchart shown in FIG. 13 shows the oxygen saturation concentration measurement method. The oxygen saturation concentration measurement method corresponds to an example of a biological information measurement method. FIG. 13 shows an oxygen saturation concentration measurement method executed by the second measurement device 100b.

In step S201, the second measurement device 100b emits the red light RL, the infrared light NL, and the green light GL. The detection control unit 31 operated in the control unit 30 causes, via the drive circuit 13, the light-emitting element 11 to emit light. The detection control unit 31 controls the light-emitting element unit 10 to emit light to the user M. The detection control unit 31 causes the light-emitting element unit 10 to emit the red light RL to the user M. The red light-emitting element 11a emits the red light RL to the user M. The detection control unit 31 causes the light-emitting element unit 10 to emit the infrared light NL to the user M. The infrared light-emitting element 11b emits the infrared light NL to the user M. The detection control unit 31 causes the light-emitting element unit 10 to emit the green light GL to the user M. The green light-emitting element 11c emits the green light GL to the user M.

After emitting the red light RL, the infrared light NL, and the green light GL, the second measurement device 100b receives the red light RL, the infrared light NL, and the green light GL in step S203. The detection control unit 31 causes the light-receiving element unit 20 to receive the red light RL, the infrared light NL, and the green light GL that passed through the user M. The first light-receiving area 21a of the light-receiving element 21 receives the red light RL and the infrared light NL reflected by the user M. The second light-receiving area 21b of the light-receiving element 21 receives the green light GL reflected by the user M.

After receiving the red light RL, the infrared light NL, and the green light GL, the second measurement device 100b generates the red light detection signal, the infrared light detection signal, and the green light detection signal in step S205. The output circuit 23 of the light-receiving element unit 20 generates the red light detection signal based on the received red light RL. The output circuit 23 generates the red light detection signal by performing processing such as analog-to-digital conversion on the light-receiving intensity data of the red light RL detected by the light-receiving element 21. The output circuit 23 generates the infrared light detection signal based on the received infrared light NL. The output circuit 23 generates the infrared light detection signal by performing processing such as analog-to-digital conversion on the light-receiving intensity data of the infrared light NL detected by the light-receiving element 21. The output circuit 23 generates the green light detection signal based on the received green light GL. The output circuit 23 generates the green light detection signal by performing processing such as analog-to-digital conversion on the light-receiving intensity data of the green light GL detected by the light-receiving element 21.

After generating the red light detection signal, the infrared light detection signal, and the green light detection signal, the second measurement device 100b calculates the oxygen saturation concentration and the correlation coefficient in step S207. The data processing unit 33 determines the pulse wave region PB using the green light detection signal. The data processing unit 33 calculates the oxygen saturation concentration using the red light detection signal in the pulse wave region PB and the infrared light detection signal in the pulse wave region PB. The data processing unit 33 calculates the first correlation coefficient, the second correlation coefficient, and the third correlation coefficient based on the red light detection signal, the infrared light detection signal, and the green light detection signal.

After calculating the oxygen saturation concentration and the correlation coefficient, the second measurement device 100b determines the oxygen saturation concentration in step S209. The data processing unit 33 calculates the evaluation value using the first correlation coefficient, the second correlation coefficient, and the third correlation coefficient. The data processing unit 33 determines the oxygen saturation concentration based on the evaluation value. The second measurement device 100b can determine the measurement accuracy of the oxygen saturation concentration by determining the oxygen saturation concentration.

The second light-emitting element unit 10b includes the green light-emitting element 11c that emits the green light GL. The second light-receiving element unit 20b receives the green light GL and generates the green light detection signal based on the green light GL. The control unit 30 calculates the evaluation value using the red light detection signal, the infrared light detection signal, and the green light detection signal.

The accuracy of the evaluation value is improved by using the green light detection signal when calculating the evaluation value.

The second light-receiving element unit 20b includes the band-pass filter 25 that extracts an AC component. The band-pass filter 25 extracts the red light AC component from the received red light RL and extracts the infrared light AC component from the infrared light NL. The control unit 30 calculates the oxygen saturation concentration using the red light AC component and the infrared light AC component.

The signal intensity of the AC component is lower than the signal intensity of the DC component. By using the AC component separated by the band-pass filter 25, the measurement accuracy of the oxygen saturation concentration is improved.

What is claimed is:

1. A biological information measurement device comprising:
a light-emitting unit including a first light-emitting element that emits red light and a second light-emitting element that emits infrared light;
a light-receiving unit configured to receive the red light emitted from the first light-emitting element and the infrared light emitted from the second light-emitting element and generate a first light-receiving signal based on the red light and a second light-receiving signal based on the infrared light; and
a controller configured to calculate an oxygen saturation concentration,
the controller is configured to
calculate the oxygen saturation concentration using the first light-receiving signal and the second light-receiving signal,
calculate correlation data using the first light-receiving signal and the second light-receiving signal, and
determine the oxygen saturation concentration based on the correlation data.

2. The biological information measurement device according to claim 1, further comprising:
a storage unit configured to store a threshold for evaluating the correlation data, wherein
the controller is configured to determine the oxygen saturation concentration by comparing the correlation data with the threshold.

3. The biological information measurement device according to claim 2, wherein
the controller
outputs the oxygen saturation concentration when the correlation data is larger than the threshold, and
deletes the oxygen saturation concentration when the correlation data is smaller than the threshold.

4. The biological information measurement device according to claim 1, further comprising:
a display unit configured to display the oxygen saturation concentration, wherein
the controller is configured to
calculate a reliability of the oxygen saturation concentration based on the correlation data, and
cause the display unit to display the reliability.

5. The biological information measurement device according to claim 1, wherein
the light-emitting unit includes a third light-emitting element that emits green light,
the light-receiving unit is configured to receive the green light and generate a third light-receiving signal based on the green light, and
the controller is configured to calculate the correlation data using the first light-receiving signal, the second light-receiving signal, and the third light-receiving signal.

6. The biological information measurement device according to claim 1, wherein
the light-receiving unit includes a filter that extracts a fluctuation component,
the filter is configured to extract a first fluctuation component from the received red light and extract a second fluctuation component from the infrared light, and
the controller is configured to calculate the oxygen saturation concentration using the first fluctuation component and the second fluctuation component.

7. The biological information measurement device according to claim 1, wherein
the controller is configured to calculate the correlation data and the oxygen saturation concentration at a predetermined time interval.

8. The biological information measurement device according to claim 7, wherein
the controller is configured to
calculate a frequency of a pulse wave from the first light-receiving signal or the second light-receiving signal, and
determine the predetermined time interval including one or more pulse waves using the frequency.

9. A biological information measurement method comprising:
emitting red light and infrared light to a subject;
receiving the red light and the infrared light passing through the subject;
generating a first light-receiving signal based on the received red light and a second light-receiving signal based on the received infrared light;
calculating an oxygen saturation concentration using the first light-receiving signal and the second light-receiving signal;
calculating correlation data based on the first light-receiving signal and the second light-receiving signal; and
determining the oxygen saturation concentration based on the correlation data.
